SEQUENCE LISTING (91 sequences)

SEQ ID NO: 1 - DNA, 23 bp, ARTIFICIAL SEQUENCE
  Chemically Synthesized
aaatagcccttgaccaggcatcc

SEQ ID NO: 2 - DNA, 24 bp, ARTIFICIAL SEQUENCE
  Chemically Synthesized
gaaataacccttgaccaggcatcc

SEQ ID NO: 3 - DNA, 31 bp, ARTIFICIAL SEQUENCE
  Chemically Synthesized
cagtgaattcgcacacgactgaggcacctcc

SEQ ID NO: 4 - DNA, 36 bp, ARTIFICIAL SEQUENCE
  Chemically Synthesized
gactgaattcaagcttttttttttttttttttttnn

SEQ ID NO: 5 - DNA, 37 bp, ARTIFICIAL SEQUENCE
  Chemically Synthesized
agtcgtcgacggagttagtttgggcagcagatccagg

SEQ ID NO: 6 - DNA, 24 bp, ARTIFICIAL SEQUENCE
  Chemically Synthesized
gaaataacccttgaccaggcatcc

SEQ ID NO: 7 - DNA, 40 bp, ARTIFICIAL SEQUENCE
  Chemically Synthesized
cagtgaattcggaagatggatacagttggtgcagcatcag

SEQ ID NO: 8 - DNA, 44 bp, ARTIFICIAL SEQUENCE
  Chemically Synthesized
atcgtctagagccaccatggagacagacacaatcctgctatggg

SEQ ID NO: 9 - DNA, 33 bp, ARTIFICIAL SEQUENCE
  Chemically Synthesized
atcgcgtacgtttgatctccagcttggtgcctc

SEQ ID NO: 10 - DNA, 44 bp, ARTIFICIAL SEQUENCE
  Chemically Synthesized
atcgtctagagccaccatgggatggagctggatctttctctttc

SEQ ID NO: 11 - DNA, 33 bp, ARTIFICIAL SEQUENCE
  Chemically Synthesized
ggtgctagctgaggagactgtgagagtggtgcc

SEQ ID NO: 12 - DNA, 43 bp, ARTIFICIAL SEQUENCE
  Chemically Synthesized
attgctagcgctgccaccatgaggtgcctagctgagttcctgg

SEQ ID NO: 13 - DNA, 30 bp, ARTIFICIAL SEQUENCE
  Chemically Synthesized
caccgtacgtttcagctccagcttggtccc

SEQ ID NO: 14 - DNA, 44 bp, ARTIFICIAL SEQUENCE
  Chemically Synthesized
atcgtctagagccaccatgaacttcgggttcagcttgattttcc

SEQ ID NO: 15 - DNA, 33 bp, ARTIFICIAL SEQUENCE
  Chemically Synthesized
gatgctagctgaggagacggtgagtgaggttcc

SEQ ID NO: 16 - DNA, 40 bp, ARTIFICIAL SEQUENCE
  Chemically Synthesized
atcgtctagagccaccatgatgagtcctgcccagttcctg

SEQ ID NO: 17 - DNA, 31 bp, ARTIFICIAL SEQUENCE
  Chemically Synthesized
caccgtacgttttatttccagcttggtcccc

SEQ ID NO: 18 - DNA, 49 bp, ARTIFICIAL SEQUENCE
  Chemically Synthesized
atcgtctagagccaccatggaatgtaactggatacttccctttattctg

SEQ ID NO: 19 - DNA, 33 bp, ARTIFICIAL SEQUENCE
  Chemically Synthesized
ggtgctagctgaggagacggtgactgaggttcc

SEQ ID NO: 20 - DNA, 400 bp, ARTIFICIAL SEQUENCE
  Chemically Synthesized
atgggatggagctggatctttcttttctcctgtcaggctgcaggtgtcctctctgaggtccagctgcaacagtctggacctgagctggtgaaacctggggcttcagtgaagatatcctgcaagacttctggatacacattcactgaatacaccatgcactgggtgaagcagagccatggaaagagccttgaatggattggaggtattaatcctaacaatggtggttctagctacaaccagaagttcaagggcaaggccacattgactgtagacaagtcctccagcacagcctacatggagctccgcagcctgacatctgaggattctgcactctattattgtgcaagaaactaccttagcgactactggggccaaggcaccactctcacagtctcctcag

SEQ ID NO: 21 - protein, 135 aa, ARTIFICIAL SEQUENCE
  Chemically Synthesized
MGWSWIFLFLLSGTAGVLSEVQLQQSGPELVKPGASVKISCKTSGYTFTEYTMHWVKQSHGKSLEWIGGINPNNGGSSYNQKFKGKATLTVDKSSSTAYMELRSLTSEDSALYYCARNYLSDYWGQGTTLTVSSA

SEQ ID NO: 22 - DNA, 394 bp, ARTIFICIAL SEQUENCE
  Chemically Synthesized
atggagacagacacaatcctgctatgggtgctgctgctctgggttccaggctccactggtgacattgtgctgacccaatctccagcttctttggctgtgtctctagggcagagggccaccatctcctgcaaggccagccaaagtgttgattatgatggtgatagttatatgaactggtatcaacagaaaccaggacagccacccaaactcctcatctatgttgcatccaatctagaatctgggatcccagccaggtttagtggcagtgggtctgggacagacttcaccctcaacatccatcctgtggaggaggaggatgctgcaacctattactgtcaacaaagtaatgaggatcctccgacgttcggtggaggcaccaagctggagatcaaac

SEQ ID NO: 23 - protein, 127 aa, ARTIFICIAL SEQUENCE
  Chemically Synthesized
METDTILLWVLLLWVPGSTGDIVLTQSPASLAVSLGQRATSQSVDYDGDSYMNWYQQKPGQPPKLLIYVASNLESGIPARFSGSGSGTDFTLNIHPVEEEDAATYYCQQSNEDPPTFGGGTKLEIKR

SEQ ID NO: 24 - DNA, 432 bp, ARTIFICIAL SEQUENCE
  Chemically Synthesized
atgaacttcgggttcagcttgattttccttgtccttgttttaaaaggtgtccagtgtgaagtgaagctggtggagtctgggggaggcttagtgaagcctggagggtccctgaaactctcctgtgcagcctctggattcactttcagtagttatggcatgtcttgggttcgccagactccagagaagaggctggagtgggtcgcatccattaataatggtggtaccacctactatccagacagtgtgaagggccgattcaccatctccagagataatgccaggaacatcctgttcctgcaaataaacagtctgaggtctgaggacacggccatgtattactgtgcaagaggctcagacagctcggcctacgtaggaatatggtactatgctctggactactggggtcaaggaacctcactcaccgtctcctca

SEQ ID NO: 25 - protein, 144 aa, ARTIFICIAL SEQUENCE
  Chemically Synthesized
MNFGFSLIFLVLVLKGVQCEVKLVESGGGLVKPGGSLKLSCAASGFTFSSYGMSWVRQTPEKRLEWVASINNGGTTYYPDSVKGRFTISRDNARNILFLQINSLRSEDTAMYYCARGSDSSAYVGIWYYALDYWGQGTSLTVSS

SEQ ID NO: 26 - DNA, 397 bp, ARTIFICIAL SEQUENCE
  Chemically Synthesized
atgaggtgcctagctgagttcctggggctgcttgtgctctggatccctggagccattggggatattgtgatgactcaggctgcaccctctgtacctgtcactcctggagagtcagtatccatctcctgcaggtctagtaagagtctcctgcatagtaatggcaacacttacttgtattggttcctgcagaggccaggccagtctcctcagctcctgatatatcggttgtccaaccttgcctcaggagtcccagacaggttcagtggcagtgggtcaggaactgctttcacactgagaatcagtagagtggaggctgaggatgtgggtgtttattactgtttgcaacatctagaatatccgctcacgttcggtggtgggaccaagctggagctgaaac

SEQ ID NO: 27 - protein, 132 aa, ARTIFICIAL SEQUENCE
  Chemically Synthesized
MRCLAEFLGLLVLWIPGAIGDIVMTQAAPSVPVTPGESVSISCRSSKSLLHSNGNTYLYWFLQRPGQSPQLLIYRLSNLASGVPDRFSGSGSGTAFTLRISRVEAEDVGVYYCLQHLEYPLTFGGGTKLELK

SEQ ID NO: 28 - DNA, 439 bp, ARTIFICIAL SEQUENCE
  Chemically Synthesized
atggaatgtaactggatacttccctttattctgtcggtaatttccggggtctactcagaggttcagctccagcagtctgggactgtgctggcaaggcctggggcttccgtgaagatggcctgcaaggcttctggctacagcttaaccagctactggatgcactgggtaaaacagaggcctggacagggtctagaatggattggtgctatttatcctggaaatagtgatagtgatactggctacaaccagaagttcaagggcaaggccaaactgactgcagtcaaatccgccagcactggatacatggagctcagtagcctgacaaatagtgatgacgactctgcggtctattactgtacaagatcttcctattactacggtactarctactatgctatggactactggggtcaaggaacctcagtcaccgtctcctcag

SEQ ID NO: 29 - protein, 141 aa, ARTIFICIAL SEQUENCE
  Chemically Synthesized
MECNWILPFILSVISGVYSEVQLQQSGTVLARPGASVKMACECSGYSLTSYWMHWVKQRPGQGLEWIGAIYPGNSDTGYNQKFKGKAKLTAVKSASTGYMELSSLTNDDSAVYYCTRSSYYYGTNYYAMDYWGQGTSVTVS

SEQ ID NO: 30 - DNA, 397 bp, ARTIFICIAL SEQUENCE
  Chemically Synthesized
atgatgagtcctgcccagttcctgtttctgttagtgctctggattcgggaaaccaacggtgatgttgtgatgacccagactccactcactttgtcggttcccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtacacgttcggaggggggaccaagctggaaataaaac

SEQ ID NO: 31 - protein, 133 aa, ARTIFICIAL SEQUENCE
  Chemically Synthesized
MMSPAQFLFLLVLWIRETNGDVVMTQTPLTLSVPIGQPASISCKSSQSLLDSDGKTYLNWLLQRPGQSPKRLIYLVSKLDSGVPDRFTGSGSGTDFTLKISRVEAEDLGVYYCWQGTHFPYTFGGGTKLEIKK

SEQ ID NO: 32 - DNA, 41 bp, ARTIFICIAL SEQUENCE
  Chemically Synthesized
gtgtcgtgaaaactacccctaaaagccaaatctagagccac

SEQ ID NO: 33 - DNA, 41 bp, ARTIFICIAL SEQUENCE; identical to SEQ ID NO: 32
  Chemically Synthesized

SEQ ID NO: 34 - DNA, 50 bp, ARTIFICIAL SEQUENCE
  Chemically Synthesized
gtgtcgtgaaaactacccctaaaagccaaatctagatcgtctagagccac

SEQ ID NO: 35 - DNA, 50 bp, ARTIFICIAL SEQUENCE
  Chemically Synthesized
catgggatggagctggatctttctctttctcctgtcaggaactgcaggtg

SEQ ID NO: 36 - DNA, 50 bp, ARTIFICIAL SEQUENCE
  Chemically Synthesized
catgaacttcgggttcagcttgattttccttgtccttgttttaaaaggtg

SEQ ID NO: 37 - DNA, 50 bp, ARTIFICIAL SEQUENCE
  Chemically Synthesized
catggaatgtaactggatacttccctttattctgtcggtaatttccgggg

SEQ ID NO: 38 - DNA, 50 bp, ARTIFICIAL SEQUENCE
  Chemically Synthesized
tcctctctgaggtccagctgcaacagtctggacctgagctggtgaaacct

SEQ ID NO: 39 - DNA, 50 bp, ARTIFICIAL SEQUENCE
  Chemically Synthesized
tccagtgtgaagtgaagctggtggagtctgggggaggcttagtgaagcct

SEQ ID NO: 40 - DNA, 50 bp, ARTIFICIAL SEQUENCE
  Chemically Synthesized
tctactcagaggttcagctccagcagtctgggactgtgctggcaaggcct

SEQ ID NO: 41 - DNA, 50 bp, ARTIFICIAL SEQUENCE
  Chemically Synthesized
ggggcttcagtgaagatatcctgcaagacttctggatacacattcactga

SEQ ID NO: 42 - DNA, 50 bp, ARTIFICIAL SEQUENCE
  Chemically Synthesized
ggagggtccctgaaactctcctgtgcagcctctggattcactttcagtag

SEQ ID NO: 43 - DNA, 50 bp, ARTIFICIAL SEQUENCE
  Chemically Synthesized
ggggcttccgtgaagatggcctgcaaggcttctggctacagcttaaccag

SEQ ID NO: 44 - DNA, 50 bp, ARTIFICIAL SEQUENCE
  Chemically Synthesized
atacaccatgcactgggtgaagcagagccatggaaagagccttgaatgga

SEQ ID NO: 45 - DNA, 49 bp, ARTIFICIAL SEQUENCE
  Chemically Synthesized
ttatggcatgtcttgggttcgccagactccagagagaggctggagtggg

SEQ ID NO: 46 - DNA, 50 bp, ARTIFICIAL SEQUENCE
  Chemically Synthesized
ctactggatgcactgggtaaaacagaggcctggacagggtctagaatgga

SEQ ID NO: 47 - DNA, 44 bp, ARTIFICIAL SEQUENCE
  Chemically Synthesized
ttggaggtattaatcctaacaatggtggttctagctacaaccag

SEQ ID NO: 48 - DNA, 41 bp, ARTIFICIAL SEQUENCE
  Chemically Synthesized
tcgcatccattaataatggtggtaccacctactatccagac

SEQ ID NO: 49 - DNA, 44 bp, ARTIFICIAL SEQUENCE
  Chemically Synthesized
ttggtgctatttatcctggaaatagtgatactggctacaaccag

SEQ ID NO: 50 - DNA, 50 bp, ARTIFICIAL SEQUENCE
  Chemically Synthesized
aagttcaagggcaaggccacattgactgtagacaagtcctccagcacagc

SEQ ID NO: 51 - DNA, 41 bp, ARTIFICIAL SEQUENCE
  Chemically Synthesized
agtgtgaagggccgattcaccatctccagagataacatcct

SEQ ID NO: 52 - DNA, 50 bp, ARTIFICIAL SEQUENCE
  Chemically Synthesized
aagttcaagggcaaggccaaactgactgcagtcaaatccgccagcactgg

SEQ ID NO: 53 - DNA, 50 bp, ARTIFICIAL SEQUENCE
  Chemically Synthesized
ctacatggagctccgcagcctgacatctgaggattctgcactctattatt

SEQ ID NO: 54 - DNA, 50 bp, ARTIFICIAL SEQUENCE
  Chemically Synthesized
gttcctgcaaataaacagtctgaggtctgaggacacggccatgtattact

SEQ ID NO: 55 - DNA, 50 bp, ARTIFICIAL SEQUENCE
  Chemically Synthesized
atacatggagctcagtagcctgacaaatgacgactctgcggtctattact

SEQ ID NO: 56 - DNA, 16 bp, ARTIFICIAL SEQUENCE
  Chemically Synthesized
gtgcaagaaactacct

SEQ ID NO: 57 - DNA, 48 bp, ARTIFICIAL SEQUENCE
  Chemically Synthesized
gtgcaagaggctcagacagctcggcctacgtaggaatatggtactatg

SEQ ID NO: 58 - DNA, 39 bp, ARTIFICIAL SEQUENCE
  Chemically Synthesized
gtacaagatcttcctattactacggtactaactactatg

SEQ ID NO: 59 - DNA, 49 bp, ARTIFICIAL SEQUENCE
  Chemically Synthesized
tagcgactactggggccaaggcaccactctcacagtctcctcagctagc

SEQ ID NO: 60 - DNA, 50 bp, ARTIFICIAL SEQUENCE
  Chemically Synthesized
ctctggactactggggtcaaggaacctcactcaccgtctcctcagctagc

SEQ ID NO: 61 - DNA, 50 bp, ARTIFICIAL SEQUENCE
  Chemically Synthesized
ctatggactactggggtcaaggaacctcagtcaccgtctcctcagctagc

SEQ ID NO: 62 - DNA, 50 bp, ARTIFICIAL SEQUENCE
  Chemically Synthesized
ggtgtcgtgaaaactacccctaaaagccaaatctagatcgtctagagcca

SEQ ID NO: 63 - DNA, 44 bp, ARTIFICIAL SEQUENCE
  Chemically Synthesized
ggtgtcgtgaaaactacccctaaaagccaaatctagcgctgcca

SEQ ID NO: 64 - DNA, 41 bp, ARTIFICIAL SEQUENCE
  Chemically Synthesized
ggtgtcgtgaaaactacccctaaaagccaaatctagagcca

SEQ ID NO: 65 - DNA, 44 bp, ARTIFICIAL SEQUENCE
  Chemically Synthesized
ccatggagacagacacaatcctgctatgggtgctgctgctctgg

SEQ ID NO: 66 - DNA, 44 bp, ARTIFICIAL SEQUENCE
  Chemically Synthesized
ccatgaggtgcctagctgagttcctggggctgcttgtgctctgg

SEQ ID NO: 67 - DNA, 44 bp, ARTIFICIAL SEQUENCE
  Chemically Synthesized
ccatgatgagtcctgcccagttcctgtttctgttagtgctctgg

SEQ ID NO: 68 - DNA, 43 bp, ARTIFICIAL SEQUENCE
  Chemically Synthesized
gttccaggctccactggtgacattgtgctgacccaatctccag

SEQ ID NO: 69 - DNA, 44 bp, ARTIFICIAL SEQUENCE
  Chemically Synthesized
atccctggagccattggggatattgtgatgactcaggctgcacc

SEQ ID NO: 70 - DNA, 44 bp, ARTIFICIAL SEQUENCE
  Chemically Synthesized
attcgggaaaccaacggtgatgttgtgatgacccagactccact

SEQ ID NO: 71 - DNA, 50 bp, ARTIFICIAL SEQUENCE
  Chemically Synthesized
ttctttggctgtgtctctagggcagagggccaccatctcctgcaaggcca

SEQ ID NO: 72 - DNA, 50 bp, ARTIFICIAL SEQUENCE
  Chemically Synthesized
ctctgtacctgtcactcctggagagtcagtatccatctcctgcaggtcta

SEQ ID NO: 73 - DNA, 50 bp, ARTIFICIAL SEQUENCE
  Chemically Synthesized
cactttgtcggttcccattggacaaccagcctccatctcttgcaagtcaa

SEQ ID NO: 74 - DNA, 44 bp, ARTIFICIAL SEQUENCE
  Chemically Synthesized
gccaaagtgttgattatgatggtgatagttatatgaactggtat

SEQ ID NO: 75 - DNA, 47 bp, ARTIFICIAL SEQUENCE
  Chemically Synthesized
gtaagagtctcctgcatagtaatggcaacacttacttgtattggttc

SEQ ID NO: 76 - DNA, 47 bp, ARTIFICIAL SEQUENCE
  Chemically Synthesized
gtcagagcctcttagatagtgatggaaagacatatttgaattggttg

SEQ ID NO: 77 - DNA, 50 bp, ARTIFICIAL SEQUENCE
  Chemically Synthesized
caacagaaaccaggacagccacccaaactcctcatctatgttgcatccaa

SEQ ID NO: 78 - DNA, 50 bp, ARTIFICIAL SEQUENCE
  Chemically Synthesized
ctgcagaggccaggccagtctcctcagctcctgatatatcggttgtccaa

SEQ ID NO: 79 - DNA, 50 bp, ARTIFICIAL SEQUENCE
  Chemically Synthesized
ttacagaggccaggccagtctccaaagcgcctaatctatctggtgtctaa

SEQ ID NO: 80 - DNA, 50 bp, ARTIFICIAL SEQUENCE
  Chemically Synthesized
tctagaatctgggatcccagccaggtttagtggcagtgggtctgggacag

SEQ ID NO: 81 - DNA, 50 bp, ARTIFICIAL SEQUENCE
  Chemically Synthesized
ccttgcctcaggagtcccagacaggttcagtggcagtgggtcaggaactg

SEQ ID NO: 82 - DNA, 50 bp, ARTIFICIAL SEQUENCE
  Chemically Synthesized
actggactctggagtccctgacaggttcactggcagtggatcagggacag

SEQ ID NO: 83 - DNA, 50 bp, ARTIFICIAL SEQUENCE
  Chemically Synthesized
acttcaccctcaacatccatcctgtggaggaggaggatgctgcaacctat

SEQ ID NO: 84 - DNA, 50 bp, ARTIFICIAL SEQUENCE
  Chemically Synthesized
ctttcacactgagaatcagtagagtggaggctgaggatgtgggtgtttat

SEQ ID NO: 85 - DNA, 50 bp, ARTIFICIAL SEQUENCE
  Chemically Synthesized
atttcacactgaaaatcagcagagtggaggctgaggatttgggagtttat

SEQ ID NO: 86 - DNA, 50 bp, ARTIFICIAL SEQUENCE
  Chemically Synthesized
tactgtcaacaaagtaatgaggatcctccgacgttcggtggaggcaccaa

SEQ ID NO: 87 - DNA, 50 bp, ARTIFICIAL SEQUENCE
  Chemically Synthesized
tactgtttgcaacatctagaatatccgctcacgttcggtggtgggaccaa

SEQ ID NO: 88 - DNA, 50 bp, ARTIFICIAL SEQUENCE
  Chemically Synthesized
tattgctggcaaggtacacattttccgtacacgttcggaggggggaccaa

SEQ ID NO: 89 - DNA, 31 bp, ARTIFICIAL SEQUENCE
  Chemically Synthesized
gctggagatcaaacgtacggtggctgcacca

SEQ ID NO: 90 - DNA, 30 bp, ARTIFICIAL SEQUENCE
  Chemically Synthesized
gctggagctgaaacgtacggtggctgcacc

SEQ ID NO: 91 - DNA, 31 bp, ARTIFICIAL SEQUENCE
  Chemically Synthesized
gctggaaataaaacgtacggtggctgcacca

(19) United States
(12) Patent Application Publication
(10) Pub. No.: US 2007/0269442 A1
(43) Pub. Date: Nov. 22, 2007

(54) Chimeric monoclonal antibody recognizing iNOS

(75) Inventors: Robert J. Webber, Las Vegas, NV (US); Douglas S. Webber, Las Vegas, NV (US); Thelma H. Dixon, Las Vegas, NV (US)

Correspondence Address: THEODORE J. BIELEN JR.;BIELEN, LAMPE, & THOEMING, 1390 WILLOW PASS ROAD, SUITE 1020, CONCORD, CA, 94520, US

(73) Assignees: Robert J. Webber; Douglas S. Webbe; Thelma H. Dixon

(21) Appl. No.: 11/437,367

(22) Filed: May 19, 2006

(51) Int. Cl. A61K 39/395; C07K 16/40

(52) U.S. Cl. 424/146.1; 530/388.26

(57) ABSTRACT

A chimeric therapeutic agent recognizing iNOS utilizing a human/mouse chimeric anti-hiNOS monoclonal antibody having mouse complimentarity-determining regions forming a binding site for iNOS.

### BACKGROUND OF THE INVENTION

The present invention relates to a novel and useful chimeric therapeutic agent recognizing iNOS in mammalian subjects.

Nitric Oxide Synthase (NOS) is an enzyme which is found in humans (iNOS the inducible form) and has been associated as indicating certain pathological disease states such as sepsis. Sepsis is estimated to kill more than 200,000 people annually in the United States alone. Notably, iNOS in the blood of a mammalian subject has been linked to the onset of sepsis, severe sepsis, and septic shock conditions in humans.

Reference is made to U.S. Pat. No. 6,531,578 in which monoclonal antibodies are described that are specific for the recognition of iNOS in humans without cross-reacting with human eNOS or nNOS. In addition, United States publication 20050281826 teaches the employment of anti-hiNOS monoclonal antibodies to remove or deplete hiNOS from LPS-primed mice and, thus, to protect such mice from the lethal effect of the particulate fraction of hiNOS.

The first monoclonal antibodies (MAbs) were developed by Kohler and Milstein in 1975 by selecting and cloning hybridomas that had been produced by somatic cell hybridization of mouse spleen cells from immunized mice with immortal mouse myeloma cells. Thus, in combination, the meyloma cell provides immortality to the hybridoma which allows it to be grown in culture indefinitely. Also, the immune B-cell from the spleen confers antigen specificity through the production of an antibody. Since the initial creation of monoclonal antibodies, many monoclonal antibodies (MAbs) have been developed and used in diagnostic tests and as therapeutic agents. It became apparent that the first mouse MAbs tested in clinical trials on humans had very limited potential as therapeutics because the human immune system recognized the mouse (murine) MAbs as “foreign” proteins, which resulted in the production of human anti-mouse antibodies (HAMAs). Once the HAMAs developed, the mouse MAbs were cleared from the circulatory system very quickly, which lowered the effectiveness of the mouse MAbs. It was also discovered early on that mouse MAbs were poor at eliciting a cellular immune response in humans through the activation of macrophages and T-cells.

Initially, researchers tried to bypass the need for any mouse component in MAbs by developing human MAbs from human immunized B-cells and human myeloma cells in a manner analogous to the procedure used to produce mouse MAbs. Unfortunately, such humanized MAbs did not work well. These failures were attributed to either the fusion process not yielding viable hybridomas, the cell line being unstable, or the human MAbs produced being of poor quality, possessing low titer and low affinity. Alternative ways to make less antigenic MAbs for human immunotherapy were developed using other techniques such as human/mouse chimeric MAbs.

The IgG class of antibodies is composed of two heavy gamma chains and two light chains (either kappa or lambda) which are held together by interchain disulfide bonds. The kappa and lambda light chains are composed of two distinct domains: a variable domain (Lv) and a constant domain (Lc). The heavy gamma chains are composed of five distinct domains: a variable domain (Hv), and four constant domains (Hc₁, Hc₂, Hc₃, and Hc₄). The Lv and Hv domains both contain three hypervariable regions in the form of hypervariable loops, which are also known as complimentarity-determining regions (CDRs). The Lv and Hv hypervariable loops are in close spatial juxtaposition and, together, form the antibody binding site. For example in human/mouse chimeric MAbs the Lv and Hv binding domains of a mouse MAb are grafted onto the constant domains of a human IgG₁ scaffold backbone. In other words, the highly antigenic Fc portion of the antibody (comprising Hc₂, Hc₃, and Hc₄) is changed from a mouse structure to a human structure. Therefore the overall antigenicity of the chimeric MAb has been lowered significantly. Moreover, in such chimeric MAbs the variable domains form the original mouse binding sites which maintain antigen binding and specificity, while the molecules antigeniticy in humans has been dramatically decreased since all the constant domains, including the highly antigenic Fc region are formed from normal human serum protein IgG₁. Of course other mammalian species may be employed as a source of the non-human variable light and heavy regions of chimeric MAb, re: rat, rabbit, camel and the like.

It has been found that chimeric human/mouse MAbs have a significantly higher success rate as therapeutics than humanized MAbs. [Reichert, J. Monoclonal Antibodies In The Clinic, Nature Biotechnology 19:819 (2001)]. Also, chimeric human/mouse MAbs are considered to be generally safe, even if they prove ineffective since the target antigen of the human/mouse MAb does not play a critical role in the pathophysiology that the therapeutic is being tested to treat.

A therapeutic agent for the treatment of an illness generating iNOS in the blood utilizing a binding entity having CDRs with specific identifiable amino acid sequences would be a notable advance in the medical field.

### BRIEF SUMMARY OF THE INVENTION

In accordance with the present invention a novel and useful therapeutic agent for the treatment of an illness in a mammalian subject generating iNOS is herein provided.

A therapeutic agent of the present invention may take the form of a human/non-human chimeric anti-hiNOS monoclonal antibody. Such antibody includes a non-human variable light region and a non-human variable heavy region. In addition, the chimeric MAb would include human constant heavy domains forming the monoclonal antibody. Such constant regions may be of a human kappa or lambda light chain and gamma heavy chain type. The non-human variable light region and the non-human variable heavy region may be selected from various mammalian species such as mouse, rat, rabbit, camel, and the like. Needless to say, the non-human variable light and non-human variable heavy regions include complimentarity-determining regions (CDRs) which bind iNOS.

In addition, the amino acid sequences of the human/non-human chimeric antibody have been determined. In the case of the IgG human/non-human chimeric antibodies the CDRs have been determined for the gamma heavy chain variable domain and the kappa light chain variable domain. Such amino acid sequences have been determined for three different anti-hiNOS MAbs each having a different non-human binding site.

Also, a therapeutic agent, in the form of a single chain antibody may be employed using such sequences i.e. without the constant domain region.

It may be apparent that a novel and useful therapeutic agent for the treatment of an illness in a mammalian subject has hereinabove been described.

It is therefore an object of the present invention to provide a therapeutic agent for the treatment of an illness in a mammalian subject generating iNOS in its blood by the generation of a human/non-human chimeric anti-hiNOS monoclonal antibody.

Another object of the present invention is to provide a therapeutic agent for the treatment of an illness in a mammalian subject generation iNOS in its blood with a human/non-human chimeric monoclonal antibody utilizing a non-human variable light region and a non-human variable heavy region.

A further object of the present invention is to provide a therapeutic agent for the treatment of an illness in a mammalian subject generating iNOS in its blood in which the heavy and light chains in the variable domains contain complimentarity-determining regions that bind iNOS.

A further object of the present invention is to provide a therapeutic agent for the treatment of illness in a mammalian subject generating iNOS in its blood utilizing a pair of trios of anti-iNOS binding entities constituting complimentarity-determining regions in combination with residues placing the complimentarity-determining regions in proper juxtaposition to interact with iNOS and to bind the same.

A further object of the present invention is to provide a therapeutic agent for the treatment of an illness in a mammalian subject generating iNOS in its blood utilizing a chimeric human/mouse monoclonal antibody in which the mouse or murine portion of the antibody contains CDRs specific to the iNOS antigen.

Another object of the present invention is to provide a therapeutic agent for the treatment of an illness in a mammalian subject generating iNOS in its blood which is safe and effective.

The invention possesses objects and advantages especially as concerns particular characteristics and features thereof which will become apparent as the specification continues.

### BRIEF DESCRIPTION OF THE SEVERAL VIEWS OF THE DRAWING

FIG. 1 is a map of the expression vector pSBYL gamma 1.

FIG. 2 is a map of the expression vector pSBYL kappa.

FIG. 3A is a gamma chain nucleotide sequence of the variable region for cell line (A) of Table 1. (SEQ ID NO: 20)

FIG. 3B is a gamma chain amino acid sequence of the variable region for cell line (A) of Table 1. (SEQ ID NO: 21)

FIG. 3C is a kappa chain nucleotide sequence of the variable region for cell line (A) of Table 1. (SEQ ID NO: 22)

FIG. 3D is a kappa chain amino acid sequence of the variable region for cell line (A) of Table 1. (SEQ ID NO: 23)

FIG. 4A is a gamma chain nucleotide sequence of the variable region for cell line (D) of Table 1. (SEQ ID NO: 24)

FIG. 4B is a gamma chain amino acid sequence of the variable region for cell line (D) of Table 1. (SEQ ID NO: 25)

FIG. 4C is a kappa chain nucleotide sequence of the variable region for cell line (D) of Table 1. (SEQ ID NO: 26)

FIG. 4D is a kappa chain amino acid sequence of the variable region for cell line (D) of Table 1. (SEQ ID NO: 27)

FIG. 5A is a gamma chain nucleotide sequence of the variable region for cell line (I) of Table 1. (SEQ ID NO: 28)

FIG. 5B is a gamma chain amino acid sequence of the variable region for cell line (I) of Table 1. (SEQ ID NO: 29)

FIG. 5C is a kappa chain nucleotide sequence of the variable region for cell line (I) of Table 1. (SEQ ID NO: 30)

FIG. 5D is a kappa chain amino acid sequence of the variable region for cell line (I) of Table 1. (SEQ ID NO: 31)

FIG. 6 is an illustration of the amino acid alignment of the variable heavy (VH) regions for the cell lines (A) (D) and (I) of Table 1. (SEQ ID NO: 21, 25, & 29)

FIG. 7 is an illustration of the amino acid alignment of the variable light (VL) region for the cell lines (A) (D) and (I) of Table 1. (SEQ IS NO: 23, 27, & 31)

FIGS. 8A-C show the aligned nucleotide sequences of the variable heavy regions of cell lines (A) (D) and (I) of Table 1. (SEQ ID NOS: 32-61)

FIGS. 9A-C show the aligned nucleotide sequences of the variable light regions of cell lines (A) (D) and (I) of Table 1. (SEQ ID NOS: 62-91)

FIG. 10 is a digital image of the SDS-PAGE analysis of purified pools of chimeric human/mouse MAbs (A) (D) and (I) of the present invention with molecular weight standards shown in lane # 1.

FIG. 11 is a chart illustrating the six day survival of mice primed with L.P.S. and administered with chemical entities four hours later.

For a better understanding of the invention reference is made to the following detailed description of the preferred embodiments of the invention which should be taken in conjunction with the above described drawings.

### DETAILED DESCRIPTION OF THE PREFERRED EMBODIMENTS OF THE INVENTION

Various aspects of the present invention will evolve from the following detailed description of the preferred embodiments thereof which should be referenced to the prior described drawings.

Three human/non-human chimeric anti-hiNOS monoclonal antibodies were produced having non-human variable light regions and non-human variable heavy regions to form the binding sites for iNOS. Specifically, three recombinant CHO-DUXB11 (rCHO) cell lines were created for the manufacture of chimeric anti-iNOS antibodies. The CHO cell line used was DHFR⁻ which is its identifiable marker. It should be realized that other recombinant cells with identifiable markers could be utilized, such as, E. coli, PER.C6, Saccharomycies and the like. Genes encoding the heavy (gamma) and the light (kappa) chains of mouse anti-iNOS monoclonal antibodies were cloned from the original mouse hybridoma cell lines and amplified by reverse transcription-polymerase chain reaction (RT-PCR). Recominant amplified cell lines that produce mouse/human chimeric anti-iNOS monoclonal antibodies were, thus, created using co-transfection technology and the DHFR⁻ chain amplification system. The productivity of all such cell lines was evaluated and the best clones for each of the three antibodies were cryopreserved.

Three hybridoma cell lines were used as DNA sources to generate the mouse variable Lv and Hv regions of the anti-iNOS antibodies. The DNA sequences coding for the variable Lv and Hc sequences were grafted onto vectors containing either the Lc or Hc constant regions of the human IgG1 molecule. DNAs encoding the chimeric anti-iNOS gamma heavy chain and kappa light chain were co-transfected into DHFR-negative CHO cells. The expression of the chimeric monoclonal antibodies was amplified by culturing the rCHO cells in the presence of methotexate. Further, the amplified rCHO cells were cloned. The amplified and cloned candidate rCHO cells that stably express the three chimeric antibodies derived from the original murine hybridomas were created. The three original murine anti-hiNOS monoclonal antibodies were designated 1E8-D8, (antibody (A)), 2D10-2H9 (antibody (D)), and 24H9-1F3 (antibody (I)), based on a panel of antibodies depicted in U.S. Pat. No. 6,531,578.

Specifically, the cloning of the heavy chain variable region and light chain variable region genes was accomplished by encoding the anti-iNOS antibodies from the mouse hybridomas into plasmids using RT-PCR amplification. This was followed by construction of transfection vectors for the anti-iNOS chimeric mouse/human heavy and light chains of IgG1. Generation of multiple different recombinant CHO stable cell pools expressing the three different anti-iNOS antibodies was then accomplished using transfection, amplification, and stable clone selection procedures. Research cell banks were then prepared from the final clones.

Subclones were then selected from each of the three chimeric monoclonal antibodies for adaption into serum free medium and into suspension cultures. Further, purification of the three antibodies was attained by affinity chromatography on Protein-A resin.

Two of the chimeric anti-hiNOS monoclonal antibodies, chimeric MAb (A) and chimeric MAb (I) were tested for there ability to neutralize the lethal activity in mice challenged by a particular fraction of cytokine induced and lysed DLD-1-5B2 cells providing the lethal particulate fraction of hiNOS.

The following examples are provided to further illustrate the present invention but are not deemed to limit the invention in any manner.

### EXAMPLE I

### Plasmid Construction/Cell Transformation

Three hybridoma cell lines secreting murine anti-hiNOS monoclonal antibodies were assigned letter codes according to Table 1.

TABLE 1
Hybridoma Cell Lines Used/U.S. Pat. No. 6,531,578

| Cell Line | Hybridoma | | |
|---|---|---|---|
| Designation | Designation | Isotype | Binding Specificity |
| (A) | 1E8-B8 | (murine | binds to peptide PS- |
| | | IgG_{1,kappa)} | 5183 |
| (D) | 2D10-2H9 | (murine | binds to whole iNOS |
| | | IgG_{2a,kappa)} | only |
| (I) | 24H9-1F3 | (murine | binds to peptide PS- |
| | | IgG_{1,kappa)} | 5166 |

### Table 2 represents the host cell and vectors employed.

TABLE 2

| Host Cell | CHO-DUXB11 |
|---|---|
| Cloning vector | pBluescript ™ (Stratagene) |
| Heavy chain | pSBYL3gamma1 (SierraBiosource, |
| expression vector | Morgan Hill, CA) |
| Light chain | pSBLY11kappa (SierraBiosource, |
| expression vector | Morgan Hill, CA) |

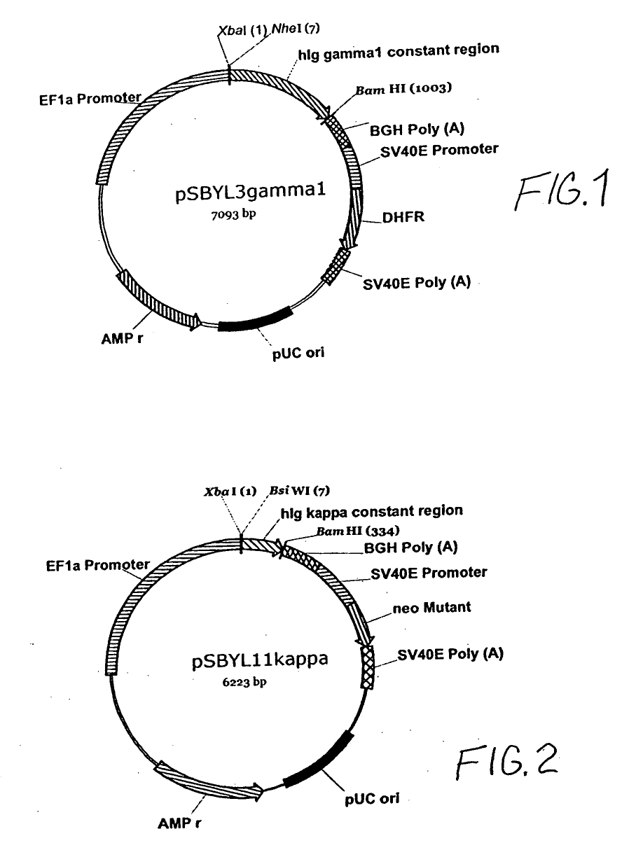

For mammalian expression, variants of expression vectors pSBYL3 and pSBYL11 were used. These variants, termed pSBYLgamma1 and pSBYL11kappa (SierraBiosource, Morgan Hill, Calif.) already contained human Igγ1 and Igκ constant regions, respectively, for convenient chimerization of mouse antibodies. Expression of target proteins in these vectors was driven by the highly effective human EF1 promoter. Selection was enabled by the dhfr gene in pSBYL3; as pSBYL11 carries a mutant of the neo marker. FIGS. 1 and 2 show the maps of the pSBYL3gamma and the SBYL11kappa expression vectors, respectively.

DMEM growth medium was used for culturing the original murine hybridoma cells. Alpha-MEM growth medium was used at all stages of rCHO cell line development work. After the addition of all components, the complete medium was filtered through a 0.22 μm filter (Stericup-GP 0.22 μm filter unit, Millipore or equivalent). For transfection selection, geneticin at 500 μg/ml was added to the medium, and for gene amplification, geneticin at 500 μg/ml and methotrexate at 50-1500 nM was added to the medium.

Total RNA from the hybridoma cells was purified using Trizol Reagent (Invitrogen, Cat No. 15596-026) according to the protocol suggested by the manufacturer with the additional step of RNA extraction with chloroform to remove traces of phenol. Spectrophotometrical RNA quantification was carried out at 260 nm assuming 1 OD to be equivalent to 40 μg/ml RNA.

The first strand of cDNA was synthesized using the Super Script III First-Strand System for RT-PCR (Invitrogen, Cat. No. 18080-051) according to the protocol suggested by the supplier. Reactions were terminated by heat inactivation for 5 min at 85° C. For RNA from each hybridoma, first-strand synthesis was primed with: 1) oligo d(T) primer from the kit, 2) primer specific for the appropriate isotype of IgG heavy chain, and 3) primer specific for Ig kappa chain. The gene specific primers used are listed below:

Heavy
Chain:

(SEQ ID NO: 1)

| | |
|---|---|
| IgG1 R_ HC: | 5′- AAATAGCCCTTGACCAGGCATCC -3′ |

(SEQ ID NO: 2)

| | |
|---|---|
| IgG2a R_{—} | 5′- GAAATAACCCTTGACCAGGCATCC -3′ |
| HC: | |
| Light | |
| Chain: | |

(SEQ ID NO: 3)

| | |
|---|---|
| MKC-R1: | 5′- CAGTGAATTCGCACACGACTGAGGCACCTCC -3′ |

The removal of RNA molecules from reverse transcription reaction was carried out by RNaseH digestion (Super Script III First-Strand System for RT-PCR) according to manufacturer's instructions. First-strand cDNA was cleaned using QIAquick PCR Purification Kit (Qiagen, Cat. No. 28706).

To facilitate PCR of first-strand cDNA with unknown 3′ sequence, poly(A) tail was appended to 3′ end of each cDNA to create a defined priming site. For this purpose, recombinant Terminal Deoxynucleotidyl Transferase (Invitrogen, Cat. No. 10533-065) was used. Reaction was carried out according to manufacturer's recommendations. Reaction product was cleaned using QIAquick PCR Purification Kit (Qiagen, Cat. No. 28706).

PCR amplification of the Ig heavy and light chain variable regions was accomplished. The primers used for the amplification of cDNA fragments of variable regions were:

Forward:

(SEQ ID NO: 4)

OligoDTF:
5′- GACTGAATTCAAGCTTTTTTTTTTTTTTTTTTTTNN -3′
Reverse, heavy chain:

(SEQ ID NO: 5)

MHCnest:
5′- AGTCGTCGACGGAGTTAGTTTGGGCAGCAGATCCAGG -3′

(SEQ ID NO: 6)

IgG2a R: HC:
5′- GAAATAACCCTTGACCAGGCATCC -3′
Reverse, light chain:

(SEQ ID NO: 7)

MKCnest:
5′- CAGTGAATTCGGAAGATGGATACAGTTGGTGCAGCATCAG -3′
for light chain.

PCR was carried out using PfuUltra High-Fidelity thermostable DNA-polymerase (Stratagene, Cat. No. 600382). Typically the first five cycles were primed only with the forward primer; annealing temperature was 45° C. After that, the reverse, gene-specific primer was added and the PCR was extended for another 30-35 cycles at annealing temperature of 50-65° C. The resulting fragments were gel purified using QIAquick Gel Extraction Kit (Qiagen, Cat. No. 28704), subcloned into pBluescript cloning vector, and sequenced.

The purified PCR products were ligated using the Quick Ligation Kit (NEB, Cat. No. M2200S) into pBluescript cloning vector cut with EcoRV. DH5α bacterial cells were transformed with the resulting DNA and spread onto LB plates supplemented with 40 μg/ml ampicillin and pre-treated with 50 μl of 20 mg/ml Xgal and 25 μl of 200 mg/ml IPTG. The colonies were blue/white selected for the presence of an insert.

Selected white colonies were picked and expanded. The DNA was isolated with QIAprep Spin Miniprep Kit (Qiagen, Cat. No. 27106). A control digest was performed with HindIII plus EcoRI. Plasmids containing the expected fragments were sequenced with T3 and T7 pBluescript-specific sequencing primers (Biotech Core, Palo Alto, Calif.).

Based on nucleotide sequences of variable regions, primers specific for the beginning of the signal peptides and for the end of J-regions were designed (Table 3). With forward primers specific for the start of translation, the Kozak motif (GCCACC), known to increase the efficiency of eukaryotic translation and XbaI or NheI restriction site, were introduced. Reverse primers specific for J-regions were designed to overlap three codons of human Ig gamma 1 and kappa constant regions and to introduce silent mutations that created NheI and SplI (BsiWI) restriction sites. Identical mutations (and restriction sites) were present at the 5′ end of genes coding for human Ig constant regions which were part of the expression vectors pSBYL3gamma1 and pSBYL11kappa of table 2. These restriction sites allowed for convenient in-frame chimerization by inserting amplified mouse heavy and light variable regions into the pSBYL3gamma1 and pSBYL11kappa plasmid backbone, respectively. The primer sequences are listed in Table 3, below.

TABLE 3

Variable Region-Specific Primers

| Antibody/ | Primer | Primer sequence |
|---|---|---|
| Chain | name | (Restriction site(s) Kozak motif) |
| A kappa | Ak-Xba_F | ATCGTCTAGAGCCACCATGGAGACAGACACAATCCTGCTA |
| ATG | | TGGG |
| | | (SEQ ID NO: 8) |
| A kappa | Ak-Spl_R | ATCGCGTACGTTTGATCTCCAGCTTGGTGCCTC |
| J1 | | (SEQ ID NO: 9) |
| A gamma | Ag-Xba_F | ATCGTCTAGAGCCACCATGGGATGGAGCTGGATCTTTCTCT |
| ATG | | TTC |
| | | (SEQ ID NO: 10) |
| A gamma | Ag-Nhe_R | GGTGCTAGCTGAGGAGACTGTGAGAGTGGTGCC |
| J2 | | (SEQ ID NO: 11) |
| D kappa | Dk- | ATTGCTAGCGCTGCCACCATGAGGTGCCTAGCTGAGTTCCT |
| ATG | Nhe/Afe_F | GG |
| | | (SEQ ID NO: 12) |
| D kappa | Dk-Spl_R | CACCGTACGTTTCAGCTCCAGCTTGGTCCC |
| J5 | | (SEQ ID NO: 13) |
| D gamma | Dg-Xba_F | ATCGTCTAGAGCCACCATGAACTTCGGGTTCAGCTTGATTT |
| ATG | | TCC |
| | | (SEQ ID NO: 14) |
| D gamma | Dg-Nhe_R | GATGCTAGCTGAGGAGACGGTGAGTGAGGTTCC |
| J4 | | (SEQ ID NO: 15) |
| I kappa | Ik-Xba_F | ATCGTCTAGAGCCACCATGATGAGTCCTGCCCAGTTCCTG |
| ATG | | (SEQ ID NO: 16) |
| I kappa | Ik-Spl_R | CACCGTACGTTTTATTTCCAGCTTGGTCCCC |
| J2 | | (SEQ ID NO: 17) |
| I gamma | Ig-Xba_F | ATCGTCTAGAGCCACCATGGAATGTAACTGGATACTTCCC |
| ATG | | TTTATTCTG |
| | | (SEQ ID NO: 18) |
| I gamma | Ig-Nhe_R | GGTGCTAGCTGAGGAGACGGTGACTGAGGTTCC |
| J4 | | (SEQ ID NO: 19) |

The XbaI site present in Ak-Xba_F and Ig-Xba_F primers was eventually not used because of an internal XbaI site in the PCR fragment. Blunt 5′ end cloning was employed instead. For this purpose, the expression vectors were digested with XbaI, the ends were filled with Klenow, and the vectors were then cut with the other restriction enzyme (NheI or SplI). The NheI site in Dk-Nhe/Afe_F primer produced ends compatible with XbaI-cut ends. Because DNA ligated in this way cannot be re-cut with either enzyme, an AfeI site was included for the purpose of control digests.

Plasmid DNA (minipreps) was isolated as above. Control digests were performed with XbaI and NheI for heavy chains and with XbaI and SplI for light chains. Two clones of each Ig chain containing the expected insert were sequenced. Plasmid DNA from final confirmed clones (one clone of each Ig chain) was prepared in large scale from 100 ml cultures with QIAfilter Plasmid Maxi Kit (Qiagen, Cat #12263) according to the manufacturer's instructions. Resulting DNA was used for transformation of DH5α bacterial cells. Bacterial cells containing the plasmid DNA were selected on LB plates with 40 μg/ml ampicillin. Viable, non-viable cell density and viability measurements were accomplished using the Trypan Blue method and a hemacytometer (Hausser Scientific, USA) or ViCell Cell Viability Analyzer.

### EXAMPLE II

### Transfection and Selection of Stable Transfectants

Transfections of CHO-DUXB11 cells were performed using Lipofectamine 2000 reagent following manufacturer's recommendation. Stable transfectants were selected using Transfectant Selection Medium containing Geneticin. Cells were plated out at approximately 70% confluency in T75 flasks in about 15 ml of Amplification Medium containing various concentrations of methotrexate from 50 to 1500 nM. Spent medium was exchanged with fresh medium every 3-5 days until colonies appeared.

Cells were seeded at 2×10⁵ viable cells per mL in 1 mL volume in 24-well plates. Cells were cultured at 37° C. with 5% CO₂ for a defined number of days (7-15). Culture medium was removed from the 24-well plates and transferred to Eppendorf tubes. The medium was centrifuged to remove cell debris. The supernatant was used for antibody measurements using IgG1-specific ELISA assay (Bethyl Human IgG ELISA Quantitation kit, Cat # E80-104).

Methotrexate was removed from the media of the most promising clones and pools of amplified cells. Cells were cultured for at least 7 weeks. Seven day quantification assays were performed to compare the production after methotrexate removal with the production of the same clone or pool which had been cultured in methotrexate.

The best clone(s) for each group (A), (D), or (I) was labeled with FITC-methotrexate and sorted on the FACSort. The brightest staining cells were collected, expanded and subcloned as described below. These subclones have the suffix “F” added to the name to indicate FACS sorting.

Six subclones of each group were selected for further expansion. These cells were expanded to 3 or 4 T-flasks. When cells had just reached confluency, they were trypsinized, counted, centrifuged, resuspended in 6 ml of freezing media, and aliquoted into 6 Nalgene cryovials. Vials were placed in Nalgene Cryo 1° C. “Mr. Frosty” Freezing Containers and frozen at −80° C. After freezing the vials were transferred to the vapor phase of liquid nitrogen freezers.

### EXAMPLE III

### Construction of Expression Vectors

The cDNAs encoding variable regions of mouse immunoglobulins were cloned into pBluescript cloning vector as outlined in Materials and Methods and sequenced. The prototype sequence of each heavy and light chain was typically determined from three clones. If the consensus sequence could not be safely determined from the first three clones, more clones were sequenced until three clones with identical full-length sequence were obtained. Repeated sequencing was frequently needed in the case of kappa chains, where the presence of aberrant kappa transcripts (originating from the hybridoma fusion partner) often created competition for the kappa templates derived from the antibody. For some antibodies, additional screening of kappa clones with RsaI restriction enzyme, allowing discriminating between aberrant and genuine transcripts, was necessary.

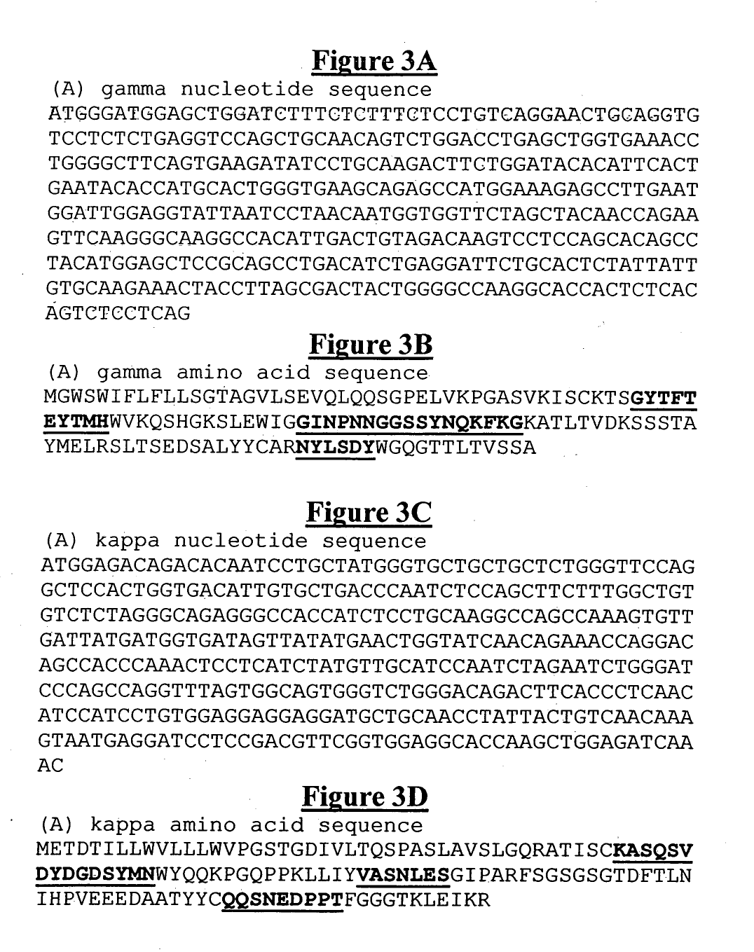

FIGS. 3-5 show coding nucleotide sequences beginning with the ATG codon of the signal peptide and ending with the J-region of the variable region or domain, along with the amino-acid translation for groups (A), (D), and (I). The three complimentary-determining regions (CDR's) contained in each variable region are denoted by bold underlined type. The spatial juxtaposition of the amino acid side chains in the 6 CDR's (three from the gamma heavy chain and three from the kappa light chain) comprise the antigen binding site for each antibody. The side chains of the MAb's amino acid residues in the CDR's bind to various functional moieties contained in the antigen (the epitope of the antigen) to form hydrogen bonds, salt bridges, hydrophobic interactions, and other noncovalent chemical bonds, and, thereby, bind specifically to the hiNOS antigen. It should be noted that conservative amino acid substitutions to the residues in the CDR's have been made by genetic engineering techniques to increase (or sometimes to decrease) an antibodies affinity constant. Further, various types of binding entities, such as single chain antibodies, mini-bodies, and others, may be employed and may incorporate an antibody's CDR's or modified CDR's to form a molecule that will bind to the same epitope as the original MAb.

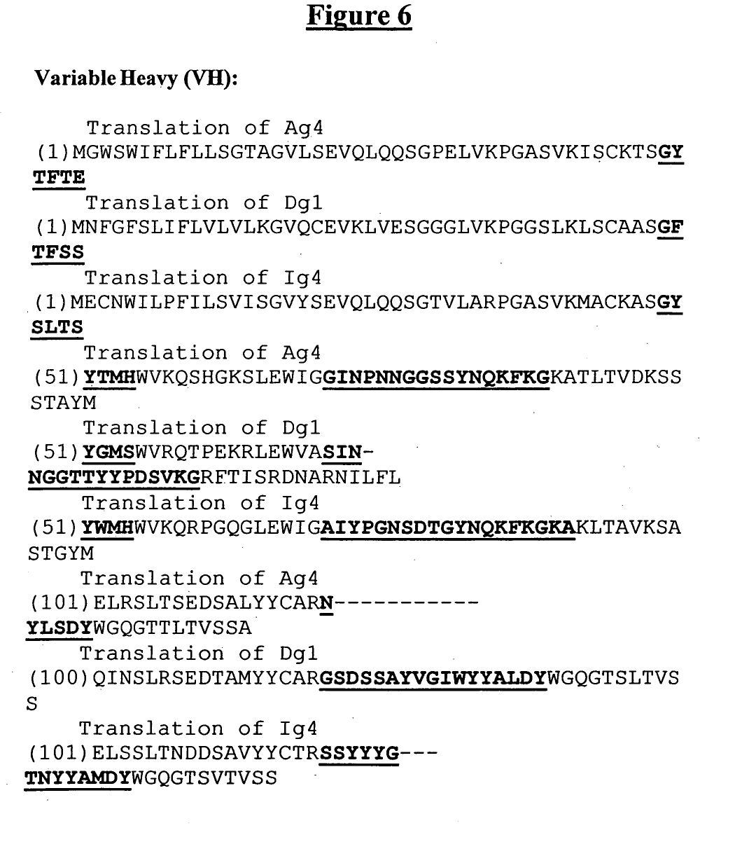

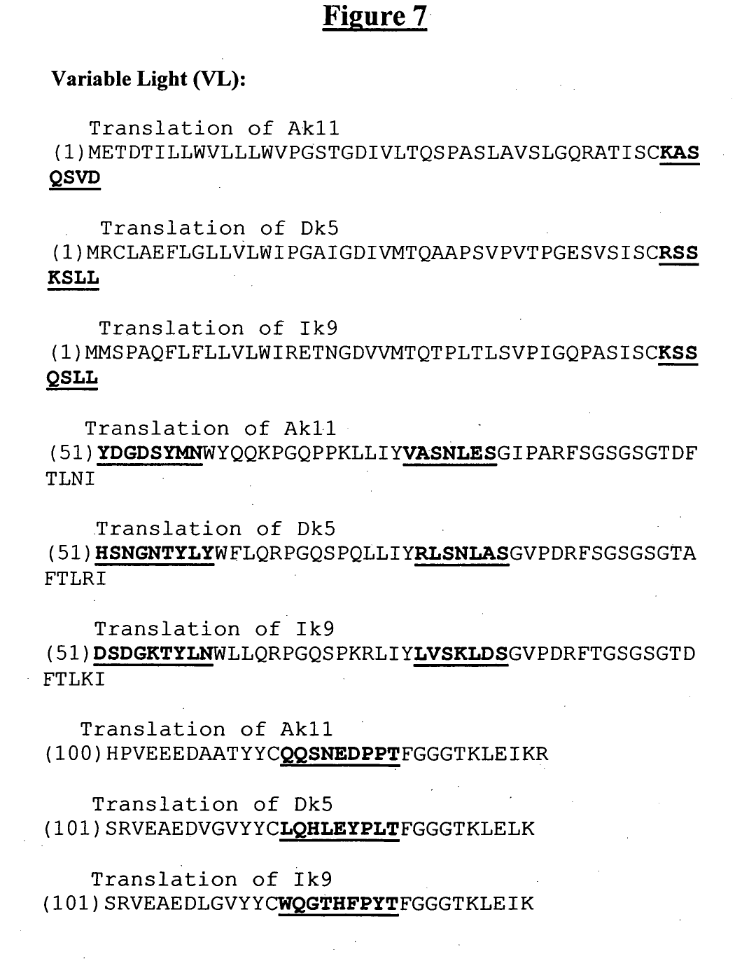

FIGS. 6 and 7 illustrate the amino acid alignment of the heavy and light variable regions.

Table 4, below, depicts exemplary conservative substitutions of amino acids in the regions of the sequences shown in FIGS. 6 and 7.

TABLE 4

| ORIGINAL | SUBSTITUTION(S) |
|---|---|
| RESIDUE | PREFERRED SUBSTITUTIONS ARE UNDERLINED |
| A (Ala) | G (Gly), v (Val), L (Leu), I (Ile) |
| C (Cys) | S (Ser) |
| D (Asp) | E (Glu), N (Asn) , Q (Gln) |
| E (Glu) | D (Asp), Q (Gln), N (Asn) |
| F (Phe) | Y (Tyr), W (Trp) |
| G (Gly) | A (Ala) |
| H (His) | A (Ala), Q (Gln), K (Lys), R (Arg) |
| I (Ile) | L (Leu), V (Val), M (Met), A (Ala), |
| | F (Phe) |
| K (Lys) | R (Arg) |
| L (Leu) | I (Ile), V (Val), M (Met), A (Ala), |
| | F (Phe) |
| M (Met) | L (Leu), I (Ile), V (Val), F (Phe) |
| N (Asn) | Q (Gln), D (Asp), E (Glu), k (Lys), |
| | R (Arg) |
| P (Pro) | A (Ala) |
| Q (Gln) | N (Asn), E (Glu), D (Asp) |
| R (Arg) | K (Lys), Q (Gln), N (Asn) |
| S (Ser) | C (Cys), T (Thr) |
| T (Thr) | S (Ser) |
| V (Val) | I (Ile), L (Leu), M (Met), F (Phe), |
| | A (Ala) |
| W (Trp) | F (Phe), Y (Tyr) |
| Y (Tyr) | F (Phe), W (Trp), T (Thr), S (Ser) |

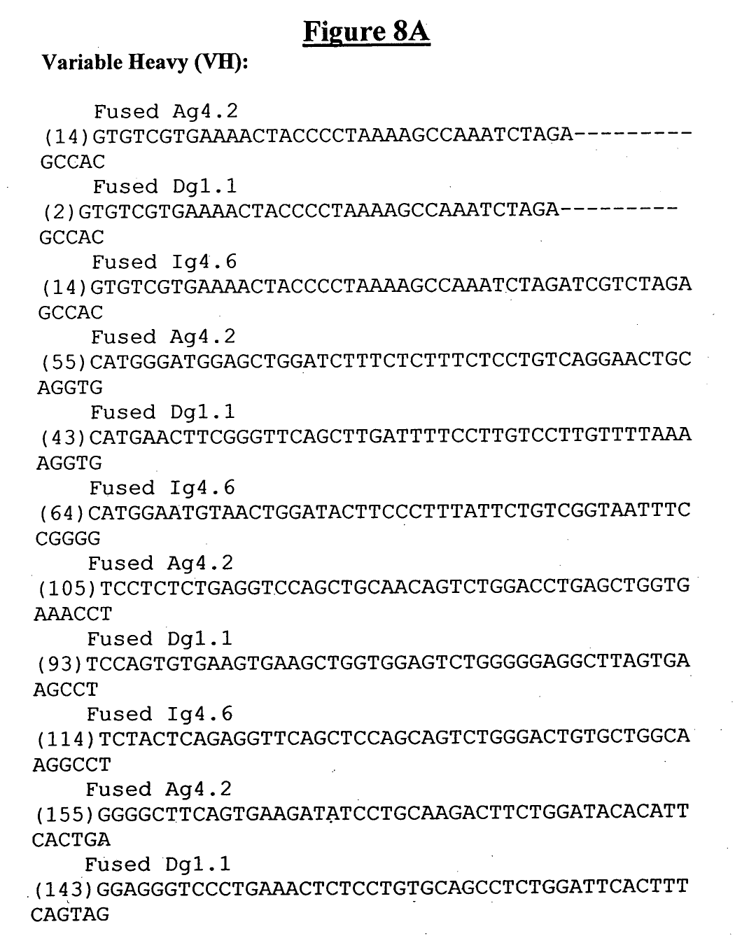

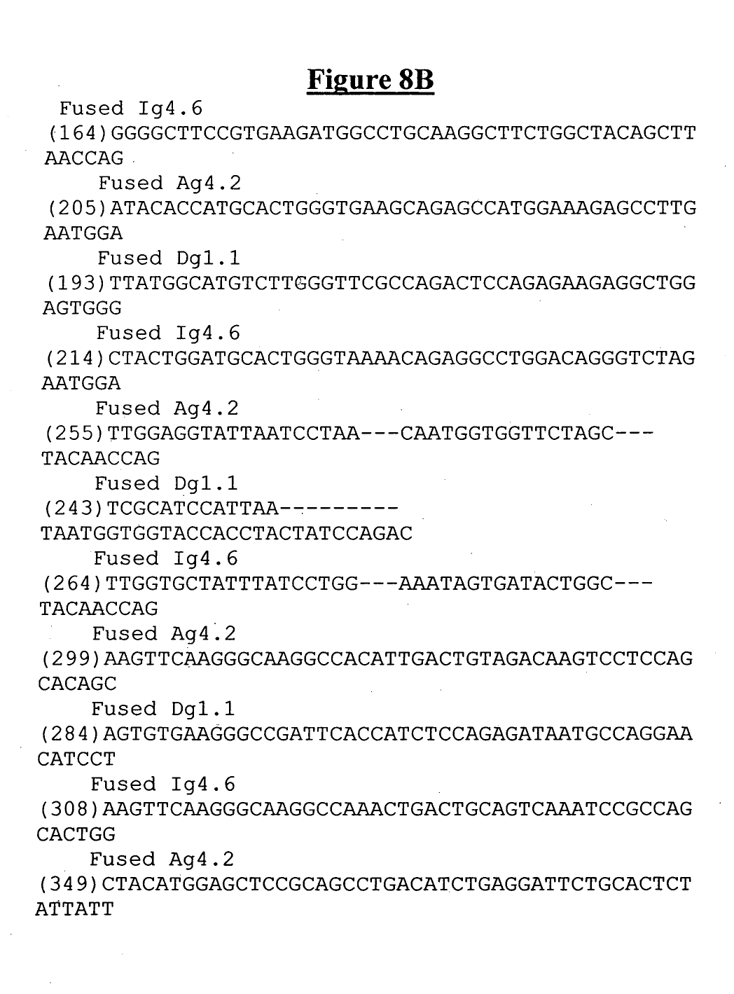

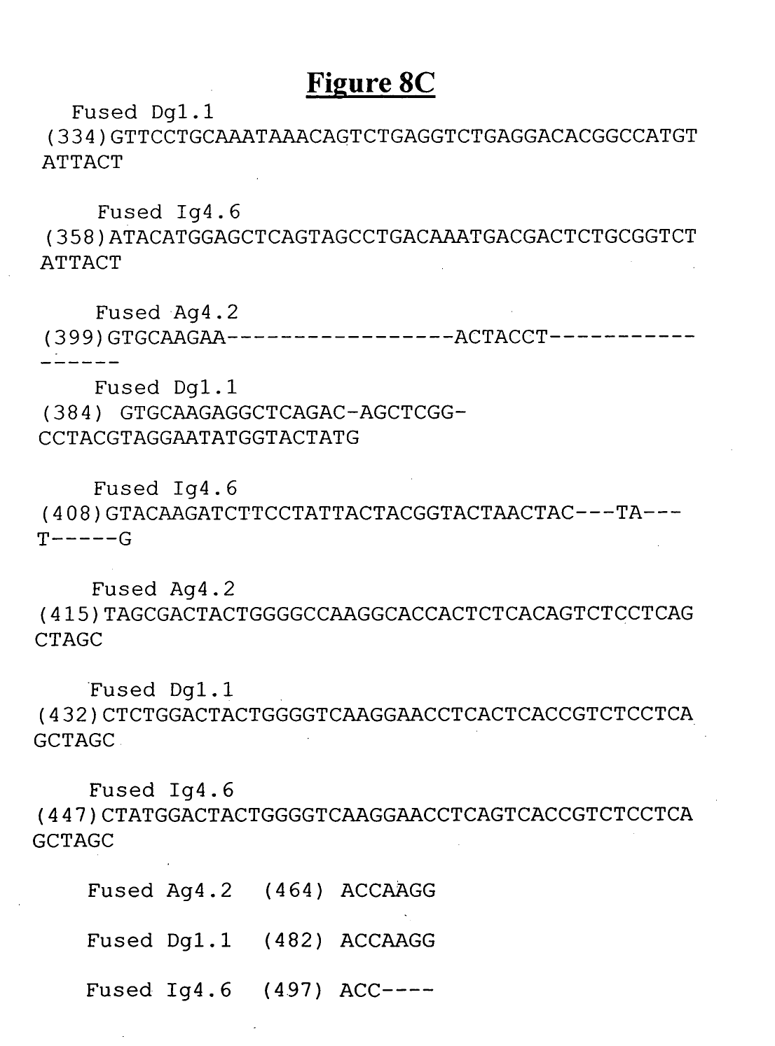

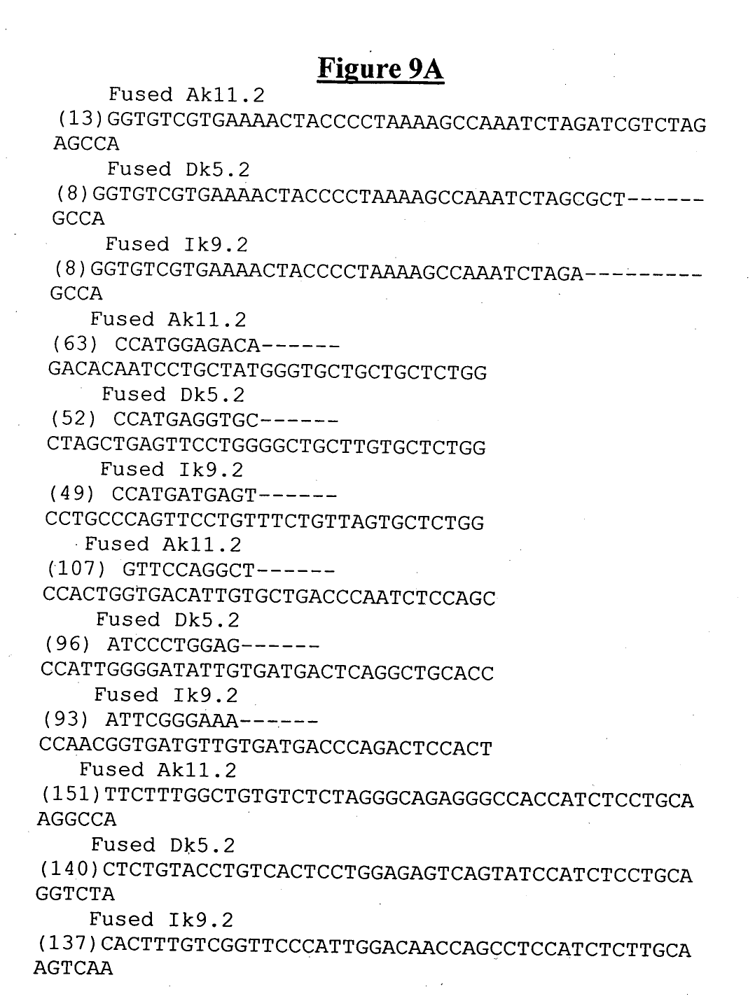

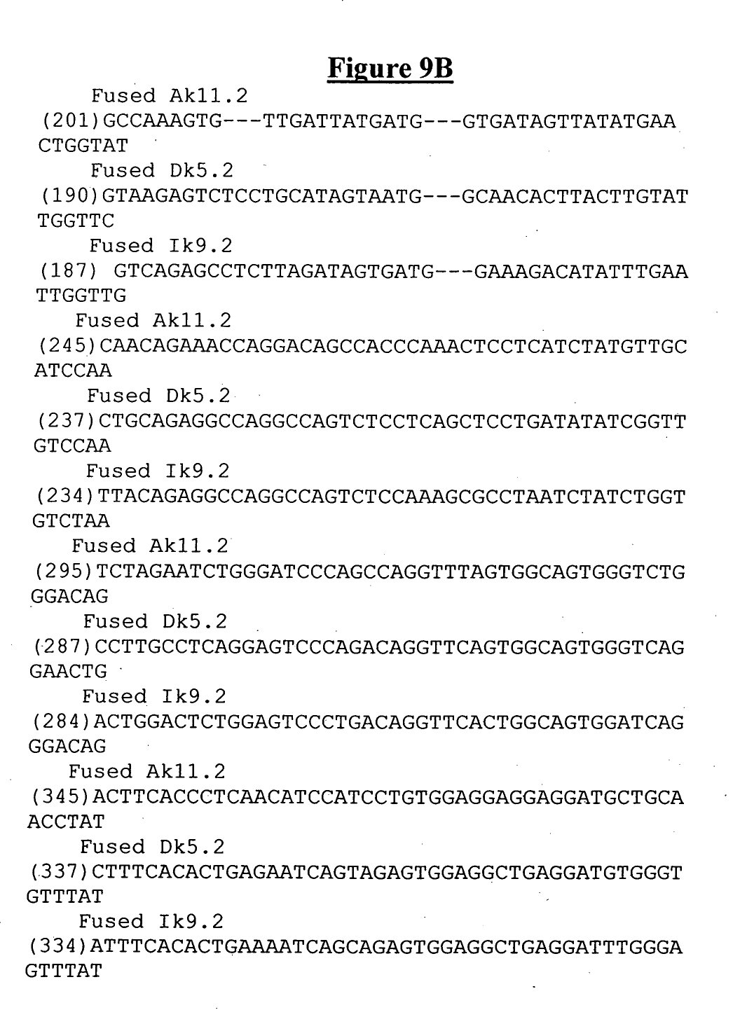

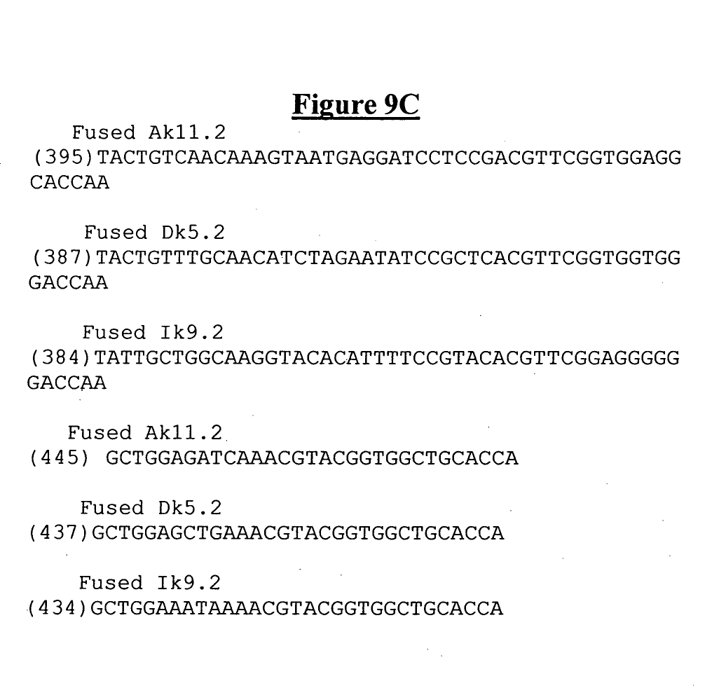

Variable regions cloned into expression vectors were verified by sequencing. FIGS. 8A-C and 9A-C show the aligned nucleotide sequences of VH and VL inserts along with the junctions and flanking vector regions.

The list of clones selected for final large-scale DNA purification are shown in Table 5 below:

TABLE 5

| Chimeric | | |
|---|---|---|
| antibody | Final HC clone | Final LC clone |
| (A) | fAg4.2 | fAk11.2 |
| (D) | fDg1.1 | fDk5.2 |
| (I) | fIg4.6 | fIk9.2 |

### EXAMPLE IV

### Generation of Cell Lines Expressing Different Chimeric Anti-iNOS Antibodies

Cell lines expressing chimeric anti-iNOS antibodies were created using the co-transfection protocol.

CHO DUXB11 cells were grown in Host Cell Growth Medium and were split every 3-4 days.

CHO-DUXB11 cells were co-transfected with expression vector DNAs coding for gamma and kappa chains of the chimeric human/mouse IgG1 using Lipofectamine 2000. Transfected cells were cultured in Host Cell Growth Medium for 1-2 days at 37° C. and 5% CO₂ prior to initiation of selection process by replacing Growth Medium with Transfectant Selection Medium.

Gene amplification was obtained by growing cells in the Amplification Medium that contained different levels of methotrexate ranging from 50 nM to 1500 nM. Two different approaches were used to amplify gene copy number: a one-step and a two-step amplification process. In one-step amplification only one level of MTX was used throughout the entire cell line development process. Two-step amplification involved two increasing MTX concentrations that were applied sequentially allowing for cell adaptation to lower level of MTX prior to subsequent cell exposure to much higher MTX level.

During the entire process of cell line development the spent medium was removed and replaced with fresh medium every 3-5 days. The MTX amplified cell pools were further expanded and their titer determined by Quantification Assay as described previously. In some cases the selected stable cell pools were subcloned prior to completion of the amplification process. Since the amplification of a single cell is shown to generate a heterogeneous cell population, these stable cell pools were subcloned again after the amplification process was completed.

The best expressing amplified pools were plated in 96 well plates to select single clones. Cells were cultured for approximately 2-3 weeks. The antibody titers of single clones were evaluated by the ELISA screening method that detects a fully assembled antibody containing two heavy gamma chains and two kappa light chains (Bethyl Human IgG ELISA Quantitation Kit, Cat# E80-104). Approximately 900-1400 clones were screened and 150-200 of the best producing clones were selected and evaluated further by additional rounds of ELISA assays.

In some cases the selected stable cell pools were subcloned prior to completion of the amplification process. The best expressing “subclones” were selected for further work. The “subclones”, that in fact became heterogeneous mini-pools (the amplification of a single cell is shown to generate heterogeneous cell population) became progenitors of a lineage from which subsequently the final fully amplified clones were generated. Likewise, “subclones” which were subjected to methotrexate removal followed by FACS sorting would have yielded a heterogeneous cell population. These were also subjected to an additional round of subcloning. Tables 6-8 list the final clones for antibodies (A), (D) and (I).

TABLE 6
Antibody (A)

| | |
|---|---|
| A-100-10C5-5D3 | One step amplification with 2 |
| | rounds of subcloning. |
| A-250-8G6-4D10 | One step amplification with 2 |
| | rounds of subcloning |
| A-250-10B11-4D9 | One step amplification with 2 |
| | rounds of subcloning. |
| A-500-1A5-5A2F | One step amplification, 1 round |
| | of subcloning, methotrexate |
| | removal, FACS sorting, 1 round |
| | of subcloning |
| A-100-10C5-4C2F | One step amplification, 1 round |
| | of subcloning, methotrexate |
| | removal, FACS sorting, 1 round |
| | of subcloning |
| A-250-10B11-1A3F | One step amplification, 1 round |
| | of subcloning, methotrexate |
| | removal, FACS sorting, 1 round |
| | of subcloning |

TABLE 7
Antibody (D)

| | |
|---|---|
| D-250-1D5-1D7 | One step amplification with 2 |
| | rounds of subcloning. |
| D-500S-5D6 | Two step amplification with 1 |
| | round of subcloning |
| D-1500-1A12 | Two step amplification with 1 |
| | round of subcloning |
| D-1500-2F10 | Two step amplification with 1 |
| | round of subcloning |
| D-250S-4A2-7C4F | Two step amplification, 1 round |
| | of subcloning, methotrexate |
| | removal, FACS sorting, 1 round |
| | of subcloning. |
| D-250S-4A2-10A10F | Two step amplification, 1 round |
| | of subcloning, methotrexate |
| | removal, FACS sorting, 1 round |
| | of subcloning. |

TABLE 8
Antibody (I)

| | |
|---|---|
| I-250-2B10-2B3 | One step amplification with 2 |
| | rounds of subcloning |
| I-250-2B10-2E3 | One step amplification with 2 |
| | rounds of subcloning |
| I-500S-3E9 | Two step amplification with 1 |
| | round of subcloning |
| I-1500-1C3 | Two step amplification with 1 |
| | round of subcloning |
| I-1500-4F9-2B10F | One step amplification, 1 round |
| | of subcloning, methotrexate |
| | removal, FACS sorting, 1 round of |
| | subcloning |
| I-1500-4F9-3F11F | One step amplification, 1 round |
| | of subcloning, methotrexate |
| | removal, FACS sorting, 1 round of |
| | subcloning |

The top clones from each group that had the highest antibody titer based on ELISA analysis were expanded from 96-well to 24-well, 6-well plates, and T75 flasks. To more accurately evaluate and rank the productivity of the selected top clones, cells were seeded at 2×10⁵ viable cell/mL in 1 mL of the Amplification Medium containing the appropriate concentration of MTX in 24-well plates. Cells were cultured for 14 days at 37° C. with 5% CO₂. The content of human IgG in the culture supernatant for each clone was measured by ELISA. Each culture supernatant was also tested for human IgG class antibodies to bind to human iNOS by EIA (all “(A)”, “(D)” and “(I)” recombinant cell line supernatants), and the culture supernatant from the “(A)” and “(I)” recombinant cell lines were tested for human IgG1 class antibody binding to synthetic peptides containing their cognate epitope sequences. The titers of the best clones selected for each of the three antibodies are summarized below in Tables 9-11. Selection of clones for cryopreservation and further developmental work was based upon the results of these tests.

TABLE 9
Best Clones for Antibody (A)

| | | | OD492 nm in |
|---|---|---|---|
| | Human IgG1 | RCU's in | Peptide |
| A | Titer | iNOS EIA at | ELISA at |
| Subclones | (ug/mL) | 1:3 dil. | 1:50 dil |
| A-100-10C5-5D3 | 37.0 | 24022 | 1.736 |
| A-250-8G6-4D10 | 52.7 | 25147 | 2.402 |
| A-250-10B11-4D9 | 21.8 | 39132 | 0.625 |
| A-500-1A5-5A2F | 14.2 | 19149 | 0.845 |
| A-100-10C5-4C2F | 15.8 | 30476 | 1.424 |
| A-250-10B11- | 9.3 | 34101 | 0.938 |
| 1A3F | | | |

TABLE 10
Best Clones for Antibody (D)

| | Human IgG1 | RCU's in |
|---|---|---|
| D | Titer | iNOS EIA at |
| Subclone | (ug/mL) | 1:4 dil |
| D-250-1D5-1D7 | 12.7 | 77132 |
| D-500S-5D6 | 16.5 | 75551 |
| D-1500-1A12 | 14.5 | 74128 |
| D-1500-2F10 | 30.2 | 70370 |
| D-250S-4A2-7C4F | 23.1 | 80875 |
| D-250S-4A2- | 14.1 | 90689 |
| 10A10F | | |

TABLE 11
Best Clones for Antibody (I)

| | | | OD492 nm in |
|---|---|---|---|
| | Human IgG1 | RCU's in | Peptide |
| I | Titer | iNOS EIA at | ELISA at |
| Subclones | (ug/mL) | 1:3 dil. | 1:50 dil |
| I-250-2B10-2B3 | 42.8 | 74941 | 0.567 |
| I-250-2B10-2E3 | 52.4 | 72435 | 0.310 |
| I-500S-3E9 | 51.1 | 87085 | 0.367 |
| I-1500-1C3 | 42.7 | 86112 | 0.569 |
| I-1500-4F9-2B10F | 36.0 | 102979 | 0.432 |
| I-1500-4F9-3F11F | 54.9 | 88240 | 0.712 |

### EXAMPLE V

### Preparation of Research Cell Banks (RCB) from Final Clones

Eighteen research cell banks were prepared. Viable cell density per vial and % viability are summarized below in table 12.

TABLE 12
Viable cell density and % viability of RCBs.

| | MTX | Date | Viable | % |
|---|---|---|---|---|
| Clone name | level | Frozen | cells/vial | viability |
| A-100-10C5-4C2F | 0 | Nov. 13, 2005 | 1.56E+07 | 97.2 |
| A-250-10B11-1A3F | 0 | Nov. 13, 2005 | 9.08E+06 | 96.5 |
| A-500-1A5-5A2F | 0 | Nov. 16, 2005 | 1.14E+07 | 97.0 |
| A-100-10C5-5D3 | 100 nM | Nov. 15, 2005 | 2.34E+07 | 98.7 |
| A-250-10B11-4D9 | 250 nM | Nov. 13, 2005 | 1.13E+07 | 96.1 |
| A-250-8G6-4D10 | 250 nM | Nov. 16, 2005 | 1.39E+07 | 97.7 |
| D-250S-4A2-10A10F | 0 | Nov. 15, 2005 | 2.17E+07 | 97.2 |
| D-250S-4A2-7C4F | 0 | Nov. 15, 2005 | 1.63E+07 | 97.8 |
| D-1500-1A12 | 1500 nM | Nov. 16, 2005 | 1.04E+07 | 97.2 |
| D-1500-2F10 | 1500 nM | Nov. 13, 2005 | 9.50E+06 | 98.0 |
| D-250-1D5-1D7 | 250 nM | Nov. 15, 2005 | 1.59E+07 | 98.2 |
| D-500S-5D6 | 500 nM | Nov. 15, 2005 | 6.67E+06 | 98.4 |
| I-1500-4F9-3F11F | 0 | Nov. 15, 2005 | 6.83E+06 | 99.1 |
| I-1500-4F9-2B10F | 0 | Nov. 15, 2005 | 1.21E+07 | 97.9 |
| I-1500-1C3 | 1500 nM | Nov. 18, 2005 | 1.21E+07 | 97.6 |
| I-250-2B10-2B3 | 250 nM | Nov. 16, 2005 | 9.67E+06 | 97.8 |
| I-500S-3E9 | 500 nM | Nov. 13, 2005 | 1.92E+07 | 98.3 |
| I-250-2B10-2E3 | 250 nM | Nov. 16, 2005 | 7.08E+06 | 95.9 |

### EXAMPLE VI

### Adaptation of Cells onto Serum Free Medium and into Suspension Culture

One subclone each of chimeric antibody (A), (D) and (I) was selected for expansion in order to perform additional experimentation with the goal of adapting each to grow and to produce chimeric human/mouse antibodies in serum free medium and in suspension culture. A cryopreserved vial each of A-100-10C5-4C2F, D-250S-4A2-7C4F and I-1500-4F9-3F11F was thawed and transferred to culture medium containing 10% fetal bovine serum (FBS). After each cell line was in log phase growth, they were transferred successively to medium containing 5%, 2.5%, 1.25%, 0.625%, 0.32% and finally to 0% fetal bovine serum. At each step the cells were allowed to return to log phase growth before being transferred to the next lower concentration of FBS. Once the cells were in log phase growth in serum-free medium, they were then adapted to grow in suspension culture by incubating the T-75 flask on an orbital platform set to 120 oscillations per minute. When the cells returned to log phase growth, they were transferred in succession to 125 ml, 500 ml, and then 2000 ml Erlenmyer shaker flasks for expansion and serum-free production of the chimeric human/mouse MAbs. Research cell banks of each cell line adapted to serum-free growth in suspension culture was cryopreserved, once expansion to a sufficient number of cells in log phase growth had been achieved (the 500 ml Erlenmyer shaker flask stage). Culture supernatant from each cell line was routinely tested for its content of human IgG1, for the ability of the chimeric (A), (D), and (I) MAbs to bind to human iNOS, and for the ability of the chimeric (A) and (I) MAbs to bind to their cognate synthetic peptide epitope. Representative data for these analytical tests are shown in Table 13.

TABLE 13
Assays of Serum-Free Supernatants from Suspension Cultures

| | | Human IgG1 | | OD 492 nm |
|---|---|---|---|---|
| Shaker | Supernatant | Titer | RCU's in | in Peptide |
| Flask | Volume | (μg/mL) | iNOS EIA | ELISA |
| A-4 | 1400 ml | 35.9 | 41738 | 1.781 |
| A-8 | 1600 ml | 41.5 | 51932 | 1.842 |
| D-2 | 700 ml | 70.1 | 37110 | N/A |
| D-5 | 1450 ml | 123.2 | 59284 | N/A |
| I-429 | 1700 ml | 51.1 | 32862 | 1.833 |
| I-431 | 1600 ml | 89.6 | 59907 | 1.862 |

### EXAMPLE VII

### Purification of Chimeric Human/Mouse Anti-iNOS MAbs (A), (D) and (I) by Protein-A Affinity Chromatography

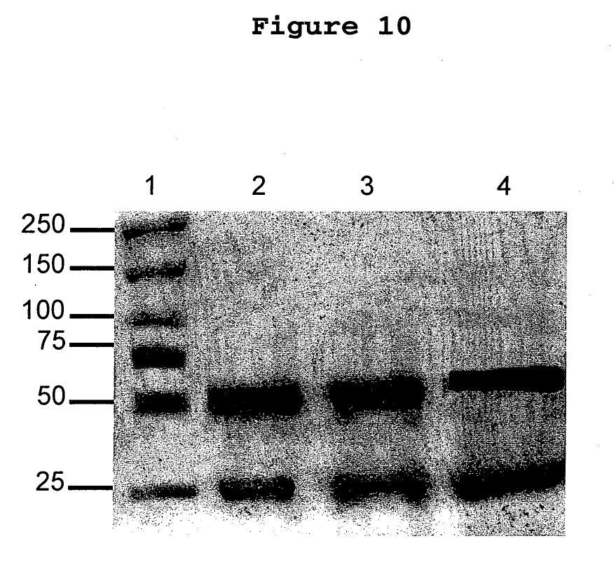

Each of the three chimeric anti-iNOS MAbs was purified by affinity chromatography using Protein-A immobilized on cross-linked agarose resin (MabSelect Media™). Each chimeric human/mouse MAbs was individually bound to the immobilized Protein-A resin. Each column was then thoroughly washed with phosphate buffered saline (PBS). The bound chimeric MAb was eluted with 0.1 M citric acid buffer pH 3.60 into tubes containing 1.0 M Tris base. Each column was then washed and stored in 20% ethanol in 0.25 M NaCl. Following application of culture supernatant, 3.0 ml fractions were collected throughout the elution process, and each fraction was analyzed for iNOS binding by EIA (A), (D) and (I), for synthetic peptide binding by ELISA (A) and (I), and for optical density at 280 nm. Fractions #6, 7 and 8 were pooled for MAb (A), fractions #5, 6 and 7 were pooled for MAb (D), and fractions #5-8 were pooled for MAb (I). The pooled MAbs were subjected to SDS-PAGE analysis using 4-20% gradient gels, after which the proteins were stained. FIG. 10 shows the SDS-PAGE of purified pools of chimeric human/mouse anti-iNOS MAbs (A), (D), and (I).

### EXAMPLE VIII

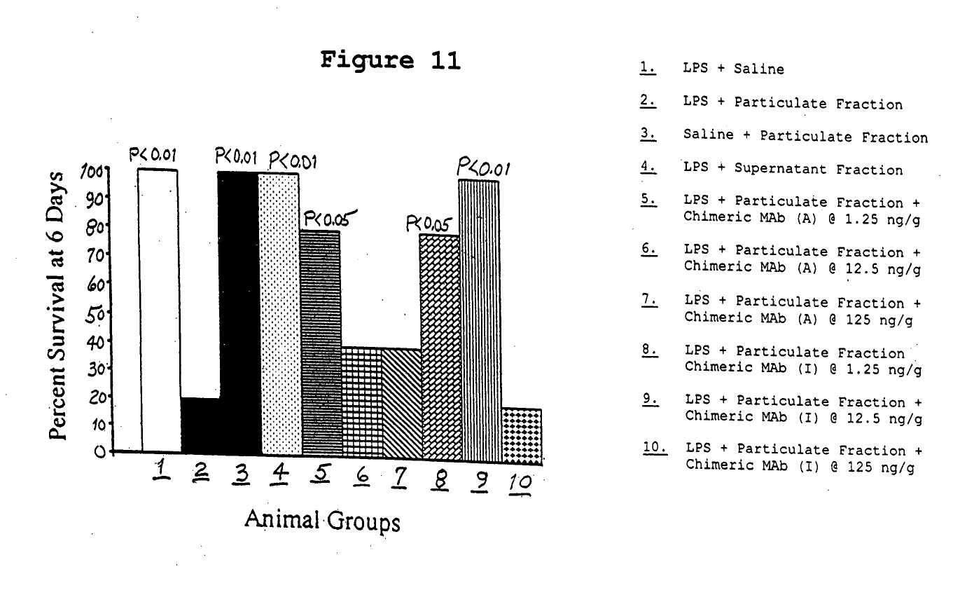

The ability of the chimeric anti-hiNOS MAbs to neutralize the lethal activity contained in the particulate fraction of cytokine induced and lysed DLD-1-5B2 cells was assessed in a series of in vivo mouse experiments. The ability of two chimeric anti-hiNOS, which originated from mouse MAb 1E-B8 ((A)) and ((I)), which originated from mouse MAb 24H9-1F3, to neutralize, and thereby to protect, LPS-primed mice from a lethal challenge of the hiNOS containing particulate fraction of cytokine induced and lysed D1D-1-5B2 cells. In these experiments, the neutralizing ability (protective effect) of these two chimeric anti-hiNOS MAbs was tested at three different doses, 1.25 ng/g body weight, 12.5 ng/g body weight, and 125 ng/g body weight. The protective effect was found to be dose-dependent. At the lower doses used in these experiments, no statistically significant difference was observed between the MAb-treated groups and the untreated group, but a trend to protect was found (FIG. 11). However, at the highest concentration of chimeric anti-hiNOS MAb (A) tested, a statistically significant difference between the MAb-treated group and untreated group was found (P<0.05 by student's T-test). Also, the protective effect of chimeric anti-hiNOS (I) was found to be statistically different at the two highest doses tested as compared to the untreated group of animals. When this MAb was used at 12.5 ng/g body weight, all 5 animals survived (p<0.01), and when used at 125 ng/g body weight, 4 of the 5 animals survived (p<0.05). These results confirm the ability of these chimeric MAbs to neutralize the lethal activity contained in the particulate fraction of cytokine induced and lysed DLD-1-5B2 cells in a dose-dependent manner by binding to the particulate hiNOS, and, thereby, sterically hindering it from binding to susceptible cells for exertion of its lethal effect(s).

While in the foregoing embodiment of the invention have been set forth in considerable detail without departing from the spirit and principals of the invention.

1. A therapeutic agent for the treatment of an illness in a mammalian subject generating iNOS in its blood, comprising:
a human/non-human chimeric anti-hiNOS monoclonal antibody having a non-human variable light region and a non-human variable heavy region forming a binding site for iNOS.

2. The therapeutic agent of claim 1 in which said non-human variable light region and non-human variable heavy region are selected from the group consisting essentially of:
mouse, rat, rabbit, camel.

3. The therapeutic agent of claim 1 in which said non-human variable light and non-human variable heavy regions include complementarity-determining regions recognizing iNOS.

4. The agent of claim 1 in which said monoclonal antibody comprises an IgG human/non-human chimeric anti-hiNOS monoclonal antibody.

5. The agent of claim 3 in which said human/non-human chimeric anti-hiNOS monoclonal antibody includes a gamma heavy chain containing said complementarity-determining regions having the amino acid sequences as disclosed in FIG. 3B.

6. The agent of claim 3 in which said human/non-human chimeric anti-hiNOS monoclonal antibody, includes a kappa light chain containing said complementarity-determining regions having the amino acid sequences as disclosed in FIG. 3D.

7. The agent of claim 6 in which said human/non-human chimeric anti-hiNOS monoclonal antibody further includes a gamma heavy chain containing said complementarity-determining regions having the amino acid sequences as disclosed in FIG. 3B.

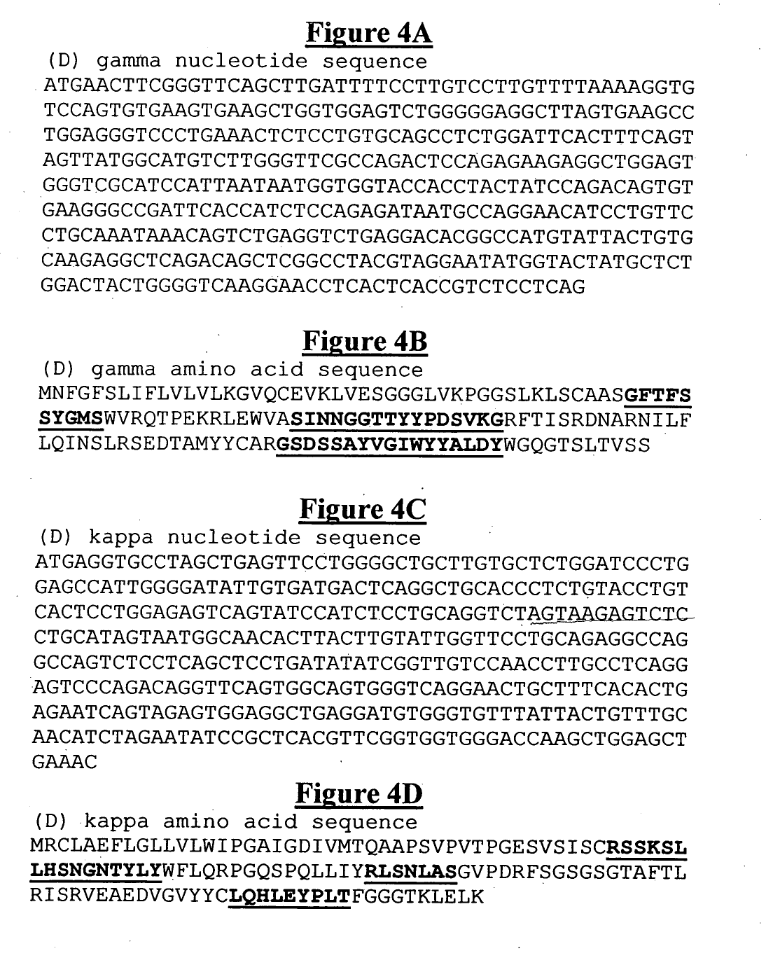

8. The agent of claim 3 in which said human/non-human chimeric anti-hiNOS monoclonal antibody includes a gamma heavy chain containing said complementarity-determining regions having the amino acid sequences as disclosed in FIG. 4B.

9. The agent of claim 3 in which said human/non-human chimeric anti-hiNOS monoclonal antibody includes a kappa light chain containing said complementarity-determining regions having the amino acid sequences as disclosed in FIG. 4D.

10. The agent of claim 9 in which said human/non-human chimeric anti-hiNOS monoclonal antibody further includes a gamma heavy chain containing said complementarity-determining regions having the amino acid sequences as disclosed in FIG. 4B.

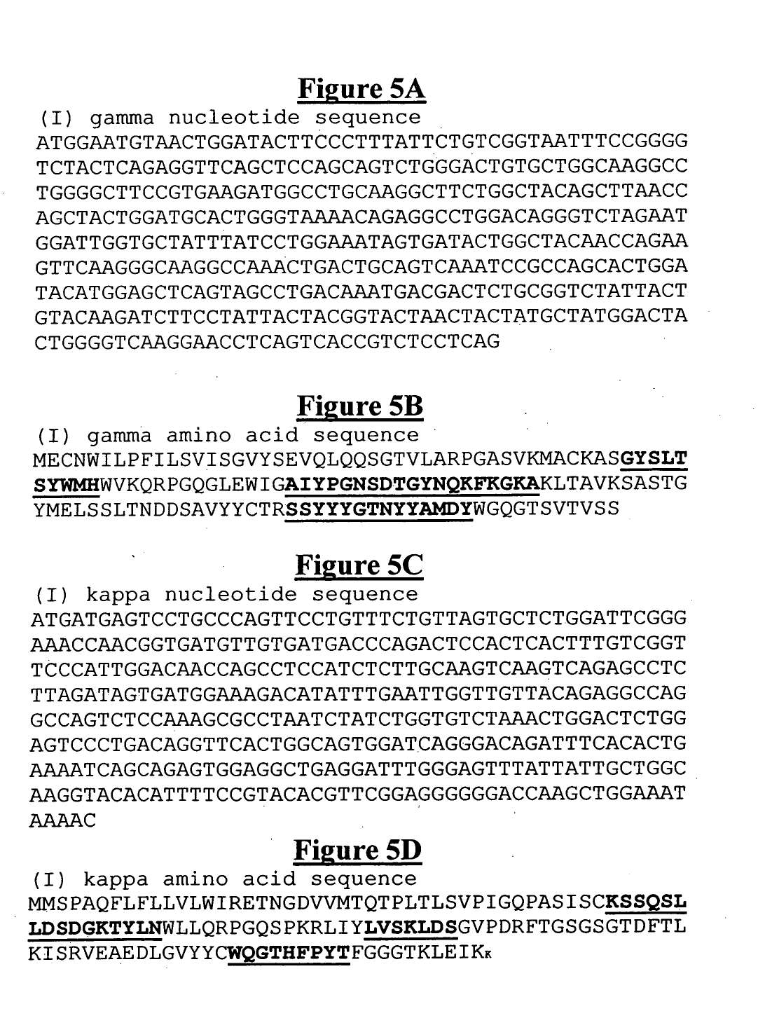

11. The agent of claim 3 in which said human/non-human chimeric anti-hiNOS monoclonal antibody includes a gamma heavy chain containing said complementarity-determining regions having the amino acid sequences as disclosed in FIG. 5B.

12. The agent of claim 3 in which said human/non-human chimeric anti-hiNOS monoclonal antibody includes a kappa light chain containing said complementarity-determining regions having the amino acid sequences as disclosed in FIG. 5D.

13. The agent of claim 12 in which said human/non-human chimeric anti-hiNOS monoclonal antibody further includes a gamma heavy chain containing said complementarity-determining regions having the amino acid sequences as disclosed in FIG. 5B.

14. The therapeutic agent of claim 1 in which said monoclonal antibody comprises:
a human/non-human chimeric anti-iNOS monoclonal antibody produced in serum-free medium having a non-human variable light region and a non-human variable heavy region forming a binding site for iNOS.

15. The therapeutic agent of claim 14 in which said human/non-human chimeric anti-iNOS monoclonal antibody produced in serum-free medium includes complimentarity-determining regions recognizing iNOS.

16. The therapeutic agent of claim 1 in which said monoclonal antibody comprises:
a purified human/non-human chimeric anti-iNOS monoclonal antibody having a non-human variable light region and a non-human variable heavy region forming a binding site for iNOS.

17. The therapeutic agent of claim 16 in which said purified human/non-human chimeric anti-iNOS monoclonal antibody includes complimentarity-determining regions recognizing iNOS.

18. The therapeutic agent of claim 1 in which said monoclonal antibody, comprises:
a human/non-human chimeric anti-hiNOS monoclonal antibody recognizing iNOS, produced in recombinant protein expression-competent cells possessing a unique identifiable marker.

19. The therapeutic agent of claim 18 in which said monoclonal antibody, comprises:
a human/non-human chimeric anti-hiNOS monoclonal antibody recognizing iNOS, produced in recombinant cells selected from the group consisting essentially of: CHO, E. coli, PER.C6, and Saccharomyces.

20. A therapeutic agent for the treatment of an illness in a mammalian subject generating iNOS in its blood, comprising:
an anti-iNOS binding entity having a first trio of complimentarity-determining regions, a second trio of complimentarity-determining regions, and residues placing said first and second trios of complimentarity-determining regions in proper juxtaposition to interact with iNOS.
